(19) United States
(12) Patent Application Publication
(10) Pub. No.: US 2018/0256086 A1
(43) Pub. Date: Sep. 13, 2018

(54) Microneedle Device for Interstitial Fluid Extraction

(71) Applicants: National Technology & Engineering Solutions of Sandia, LLC, Albuquerque, NM (US); STC.UNM, Albuquerque, NM (US)

(72) Inventors: Ronen Polsky, Albuquerque, NM (US); Philip R. Miller, Albuquerque, NM (US); Justin T. Baca, Albuquerque, NM (US)

(21) Appl. No.: 15/913,629

(22) Filed: Mar. 6, 2018

Related U.S. Application Data
(60) Provisional application No. 62/468,505, filed on Mar. 8, 2017.

(51) Int. Cl. A61B 5/145; A61B 5/15

(52) U.S. Cl. CPC A61B 5/14514; A61B 5/150022; A61B 5/150396; A61B 5/150312; A61B 5/150412; A61B 5/150984; A61B 5/150503; A61B 5/150068

(57) ABSTRACT

A microneedle device comprising a hollow microneedle protruding from the rim of an outer open holder can be used for the extraction of interstitial fluid (ISF). Dermal ISF can be extracted with the microneedle device with minimal pain and no blistering for human subjects. Extracted ISF volumes are sufficient for determining transcriptome and proteome signatures. Similar profiles in ISF, serum, and plasma samples, suggest that ISF can be a proxy for direct blood sampling. This minimally-invasive microneedle device enables real-time health monitoring applications using extracted ISF.

### CROSS-REFERENCE TO RELATED APPLICATION

This application claims the benefit of U.S. Provisional Application No. 62/468,505, filed Mar. 8, 2017, which is incorporated herein by reference.

### STATEMENT OF GOVERNMENT INTEREST

This invention was made with Government support under Contract No. DE-NA0003525 awarded by the United States Department of Energy/National Nuclear Security Administration. The Government has certain rights in the invention.

### FIELD OF THE INVENTION

The present invention relates to the extraction of bodily fluids and, in particular, to the extraction of interstitial fluid using a hollow microneedle device.

### BACKGROUND OF THE INVENTION

Standard clinical testing typically involves collecting biological fluid samples such as blood, urine, sweat, saliva, and sputum for laboratory analysis. See R. A. McPherson and M. R. Pincus, Henry's Clinical Diagnosis and Management by Laboratory Methods E-Book, Elsevier Health Sciences (2017). With the growing need for non-invasive sampling and real-time physiological monitoring, interest in exploring the skin as a reservoir of information has grown in recent years. See K. Orro et al., Biomark. Res. 2, 20 (2014); M. Portugal-Cohen and R. Kohen, Methods 61, 63 (2013); and D. Falcone et al., Skin Res. Technol. Off. J. Int. Soc. Bioeng. Skin ISBS Int. Soc. Digit. Imaging Skin ISDIS Int. Soc. Skin Imaging ISSI 23, 336 (2017). The mammalian dermis is the largest organ system in the body and forms the major barrier between the body and potentially harmful chemical and biological agents in the environment. The extraction of dermal interstitial fluid (ISF) potentially enables minimally invasive monitoring of biomarkers and medical diagnosis. The benefits of analyzing ISF include directly monitoring the tissue concentrations of unique biomarkers (e.g., proteins, nucleotides, small molecules, exosomes, and other cell-to-cell signaling species) which may not circulate in blood or be easily accessed in other body fluids. ISF also has a high concentration of immune system cells which makes the production of certain biomarkers appear in the skin, possibly even before they can be detected in the blood. This makes direct monitoring of dermal tissues and ISF an invaluable source of information for health monitoring. Additionally, ISF is a much simpler matrix than blood or plasma due to the absence of interfering agents such as red blood cells, clotting factors, and serum albumin. In particular, ISF samples may not require pre-processing and may enable analytical methods with higher signal-to-noise ratios. However, there is a paucity of knowledge on the presence of useful physiological markers in ISF. Numerous publications have attempted to elucidate the biomolecular content of dermal ISF without wide agreement on contents, particularly with respect to protein markers. See M. J. Herfst and H. van Rees, Arch. Dermatol. Res. 263, 325 (1978); S. Kayashima et al., Am. J. Physiol. 263, H1623 (1992); A. L. Krogstad et al., Br. J. Dermatol. 134, 1005 (1996); S. Mitragotri et al., J. Appl. Physiol. Bethesda Md. 1985 89, 961 (2000); and G. Rao et al., Pharm. Res. 10, 1751 (1993).

Further, minimally-invasive collection of ISF has proved challenging. Previous extraction methods (i.e. suction blister, effusion, dialysis, or sonication) may alter the composition of ISF, due to the local trauma caused by the extraction process. For instance, the suction blister fluid (SBF) method likely causes extensive cell lysis, destabilization of the stratum corneum, and separation of the dermal layers. See U. Kiistala, J. Invest. Dermatol. 50, 129 (1968). Additionally, previously reported extraction methods do not appear to be compatible with practical real-time monitoring of physiological changes.

Microneedle-enabled ISF extraction has been proposed for minimally invasive monitoring and diagnostic applications. See P. R. Miller et al., J. Mater. Chem. B 4, 1379 (2016), which is incorporated herein by reference. The advantage of microneedles versus traditional hypodermic needles is that they do not reach the nerve endings of vasculature within the dermis and therefore can be painless and minimally invasive. While microneedles provide very precise skin penetration, extraction of sufficient ISF (10-20 μl) for transcriptomic or proteomic analysis has not been reported. See E. V. Mukerjee et al., Sens. Actuators Phys. 114, 267 (2004); P. M. Wang et al., Diabetes Technol. Ther. 7, 131 (2005); and E. Eltayib et al., Eur. J. Pharm. Biopharm. Off. J. Arbeitsgemeinschaft Pharm. Verfahrenstechnik EV 102, 123 (2016). Further, microneedle insertion and ISF extraction is complicated by the dynamic properties and elasticity of skin. Stretching and tenting of skin impedes the placement of single and arrayed microneedles. See O. Olatunji et al., J. Pharm. Sci. 102, 1209 (2013); and R. F. Donnelly et al., J. Controlled Release 147, 333 (2010). After skin puncture, dermal compaction around the microneedle insertion site is believed to increase fluidic resistance in drug delivery studies. Wang et al. showed that reducing the amount of dermal compaction results in higher flow rates during drug delivery. See P. M. Wang et al., J. Invest. Dermatol. 126, 1080 (2006). Many groups have attempted ISF collection with microneedles, but limited volumes (<2 μl) were collected, limiting characterization and analysis. See E. V. Mukerjee et al., Sens. Actuators Phys. 114, 267 (2004); P. M. Wang et al., Diabetes Technol. Ther. 7, 131 (2005); and H. Chang et al., Adv. Mater. 29, 1702243 (2017).

The present invention provides a microneedle device that can minimize dermal compaction at the insertion site(s), allowing extraction of higher ISF volumes.

### SUMMARY OF THE INVENTION

The present invention is directed to a microneedle device for extracting interstitial fluid from an animal, comprising a hollow microneedle having a distal end and a beveled tip for penetration of a skin; and an outer holder having an open end with a rim separated from the inner hollow microneedle by an annular open space and wherein the beveled tip of the hollow microneedle protrudes beyond the rim of the outer holder and wherein the rim can press against the skin thereby enabling extraction of interstitial fluid from beneath the skin through the penetrating beveled tip of the hollow microneedle. A capillary tube can be attached to the distal end of the hollow microneedle for collection of the extracted ISF. Arrays of such hollow microneedle devices can be used to extract large quantities (e.g. up to 20 μl and 60 μl from humans and rats, respectively) of dermal ISF, with no need for blistering of the skin. ISF can be extracted in volumes sufficient for common downstream analyses, such as transcriptomic and proteomic profiling, and exosome isolation. The transcriptomic and proteomic content of the dermal ISF that is very similar to serum and plasma. ISF has been found to be enriched in exosomes, which have increasingly been shown to be effective for liquid biopsy applications. The isolation of these biomarkers from blood is difficult due to its complicated matrix, making ISF an intriguing substitute. Therefore, ISF can provide an informative proxy for blood in health monitoring, and microneedle-enabled sampling can provide wearable, real-time sensing devices.

### BRIEF DESCRIPTION OF THE DRAWINGS

The detailed description will refer to the following drawings, wherein like elements are referred to by like numbers.

FIG. 1A is a schematic illustration of a conventional planar microneedle substrate. FIG. 1B is a schematic illustration of a microneedle device comprising an inner hollow microneedle and a concentric outer cylindrical holder having a circular rim.

FIG. 2A is a schematic illustration of a pen needle can be inserted into a protective outer needle cap. FIG. 2B is a photograph of an assembled device showing a hollow microneedle protruding from the circular rim of a trimmed needle cap.

FIG. 3A is a photograph of a linear array of microneedle assemblies. FIG. 3B is a photograph of a microneedle array attached to the forearm for the extraction of ISF from a human.

FIG. 4 is a photograph of an imprint left after using a microneedle device of the present invention for ISF extraction.

FIG. 5A is a Venn diagram showing the distribution of 3506 proteins identified in plasma, serum and ISF of donor #1, in which 3270 proteins are in common. FIG. 5B is a Venn diagram showing the complete proteome distribution of donors 1, 2, and 3. Overall, 89% of the proteins were consistently detected in all three examined donors.

### DETAILED DESCRIPTION OF THE INVENTION

While needles (including microneedles) have been suggested to acquire ISF, prior devices either cause damage to tissues or have not been consistently successful. The present invention is directed to a simple and facile method and device to extract ISF using microneedles. The microneedle device comprises a needle holder geometry that facilitates extraction of ISF from the interstitial region beneath the skin.

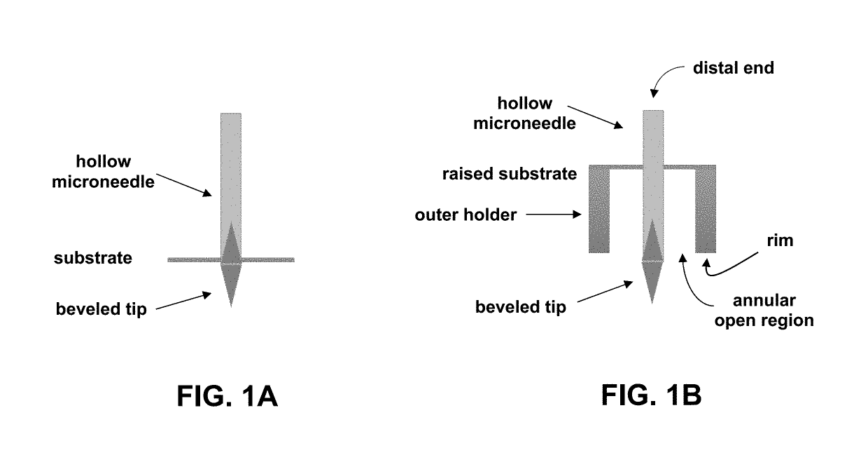

FIGS. 1A and 1B compare to two needle designs that have been tried in human subjects, a conventional flat planar geometry and a raised geometry of the present invention. The flat geometry illustrated in FIG. 1A with a hollow needle extending out-of-plane from a flat planar substrate has not been successful in extracting ISF from human subjects. In use, the flat planar substrate presses down on the skin is such a way that it pushes fluid away from the needle, thereby precluding ISF flow through the beveled opening of the hollow microneedle when it is inserted into the skin. ISF extraction has been achieved when using the raised geometry shown in FIG. 1B, where an outer hollow holder extends from a raised substrate, forming a cupped structure with an open end that provides an annular open region or void between the hollow microneedle and the rim of the holder such that the skin immediately surrounding the needle is not compressed. The penetration of the skin can be controlled by the portion of the microneedle that protrudes from the rim of the outer holder. When pressed against the skin, the concentric outer rim acts to contain the fluid in the annular region surrounding the needle, enabling extraction of the ISF through the beveled open tip of the needle. This configuration may also push fluid towards the microneedle from the pressure induced by the rim pressing against the elastic skin.

As is well known in the art, the microneedle can be made from a variety of rigid materials, including metals, ceramics, glass, silicon, and polymers and can have a variety of beveled tip geometries. The portion of the microneedle that protrudes from the rim of the holder and penetrates the skin can typically be 0.5 to 2 mm in length. The cupped holder can further be modified—e.g., the rim can be a square, triangle, etc.—and does not need to be continuous; e.g., it can have breaks and open areas. The inner diameter of a circular rim can typically be 1 to 5 mm. However, the spacing of the open annulus between the needle and holder rim can be optimized and further varied for optimal performance.

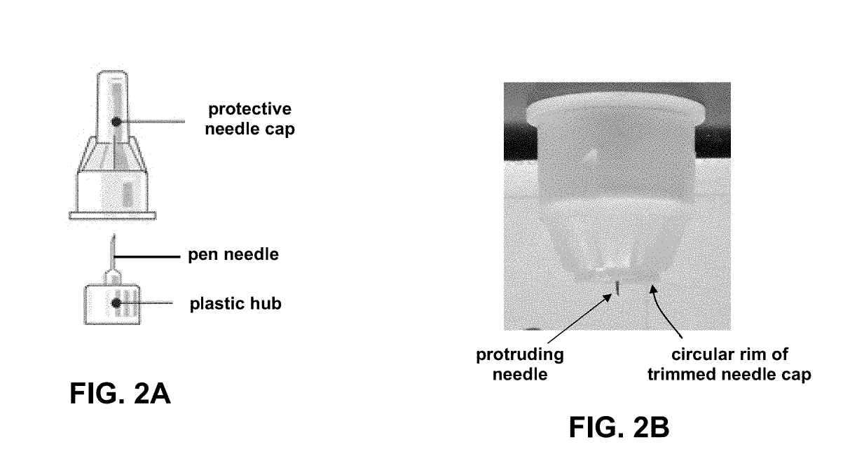

FIG. 2A is a schematic illustration of a commercial sterile pen needle package comprising a needle attached to a plastic hub that can be inserted into a protective polymer needle cap. FIG. 2B is a photograph of an assembled device showing the penetrating portion of a needle protruding from the trimmed end of the needle cap. The trimmed needle cap thereby provides an outer cylindrical holder having a circular rim for the inner hollow microneedle. Repackaging the needle within the trimmed cap allows for a controlled portion of the needle to exit the open end of the trimmed cap.

As an example of the invention, single microneedles with defined lengths were created using a CO₂ laser cutter and a three-axis stage to cut the protective plastic cap of a commercial pen needle that can be used for spaced microneedle geometries. Pen needles come in a variety of needle lengths and diameters (gauge) and are used by health professionals and patients for injection of a variety of medications, such as insulin for diabetics. As received, a single pen needle is sterile packaged and comprises a hollow needle attached to a plastic hub and protective polymer needle cap. A 32 G Ultrafine Nano Pen Needle (Becton Dickinson, Franklin Lakes, N.J.) was used in this example. The original length of the pen needle was 4 mm and needed to be shortened to be used as an insertable microneedle. The length of the insertable portion of the pen needle was controlled by trimming the protective needle cap with a CO₂ laser cutting system and reassembling the components such that the pen needle exited the open end of the trimmed portion of the cap. Precise control over pen needle insertion length was performed by adjusting the location of the laser cutter on the x-axis of the stage prior to cutting of the cap.

Three different microneedle lengths (1000 μm, 1500 μm, and 2000 μm) were initially studied for their ability to extract fluid from a human forearm with minimal pain response. In a pilot study, ISF extraction was successful in 4 of 7 human subjects. Fluid was extracted with each microneedle length, with 1500 μm needles providing a higher percentage of extraction success compared to the other lengths. For each needle length, pain scores on insertion were recorded (pain scale of 0-10 with 0 indicating absence of pain, 1 being mild irritation, and 10 being severe pain). Scores of 0.0±0.0, 0.21±0.49, and 0.71±1.11 were reported for the 1000 μm, 1500 μm, and 2000 μm microneedle lengths, respectively. A length of 1500 μm was therefore selected for subsequent studies of arrayed microneedles.

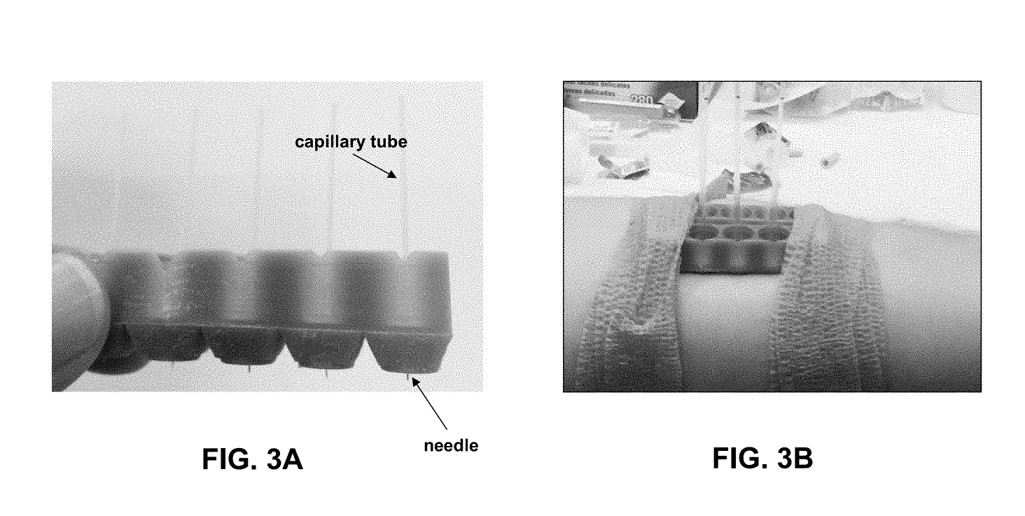

FIG. 3A is a photograph of a microneedle array comprising and array of microneedle devices within a 3D printed microneedle holder with glass capillary collection tube attached to the distal end of each microneedle. Arrays of 5 microneedles were made by 3D printing needle holders (lx 5 needle configuration) made from an acrylic resin. This number of needles allowed easy handling of the arrays for insertion. The concentric design of the needle holder was maintained for each needle. In this example, the microneedles were 1500 μm in length and the inner diameters of the concentric hollow cylinders surrounding each microneedle was ˜2.8 mm. Typically, the microneedles can be 0.5 to 2 mm in length and 1 to 400 μm in diameter. The holder can be made of a variety of materials, including polymers and metals.

FIG. 3B is a photograph of two 3D microneedle holders attached to the forearm of a human subject for ISF extraction and collection in glass capillary tubes. The 3D-printed microneedle-array holders were sterilized prior to use with ethyl alcohol, and skin was cleansed with isopropyl alcohol swabs prior to array application. The microneedle array was gently pressed against the forearm with subjects in a seated or supine posture, and held in place for the duration of sample collection. The needles can be held in place either by fixing with surgical wraps (as shown) or by hand. Insertion depth, controlled by the microneedle array holders, was 1500 μm. Arrays remained in place for up to 30 minutes while the ISF sample was collected. The microneedle array was then withdrawn, the ISF was recovered from its capillary tubes into a microcentrifuge tube on ice, and a new array was applied for another 30 minutes. Using the 5-microneedle array, up to 16 μl of ISF was extracted in 1- to 2-hour periods in human subjects. This represents a 4-5 increase in extraction efficiency over previous attempts which report on average ˜1 μl for 30 min of extraction. Overall, the success rate of ISF extraction was 92.9%, and more than 10 μl was extracted in 64% of subjects. This improvement in success rate is likely due to multiple needles being used, thus increasing the likelihood of ISF being successfully extracted each time. In addition, the spaced, concentric microneedle holder may enhance ISF flow through the microneedles by modifying local hydrostatic pressure. No observed blockage of the needle pores was observed visually after removal, and needles remained open to fluid flow with syringe testing. The extracted ISF is clear (no red blood cells) and contains no detectable cellular components. In contrast to other methods used to collect dermal interstitial fluid (i.e. blister and dialysis), no additional instrumentation is necessary (i.e. no syringe pump).

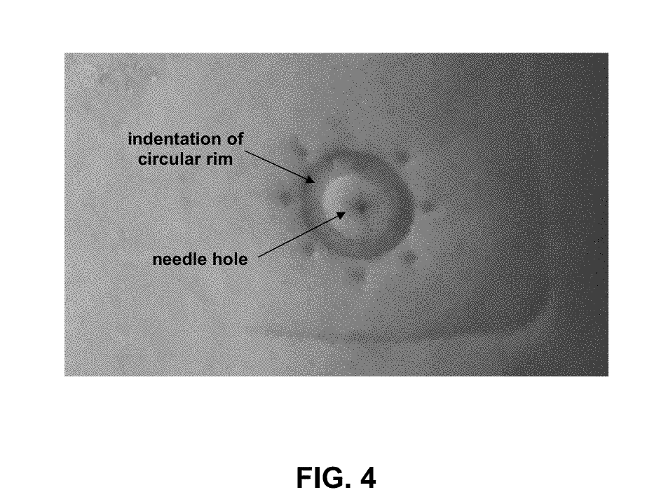

Pictured in FIG. 4 is the imprint left on the skin after extracting IF using the device shown in FIG. 3A. In the center of the circle in FIG. 4 is the single point needle hole left after extraction. There is an obvious annular “moat” of untouched skin in-between the circular indentation made by the rim of the outer cylindrical holder. This holder configuration allows ISF to remain in the skin (i.e., trapped in the “moat” region) and, moreover, the indentation produces a “pinching” effect that facilitates the flow of ISF into the open needle bevel.

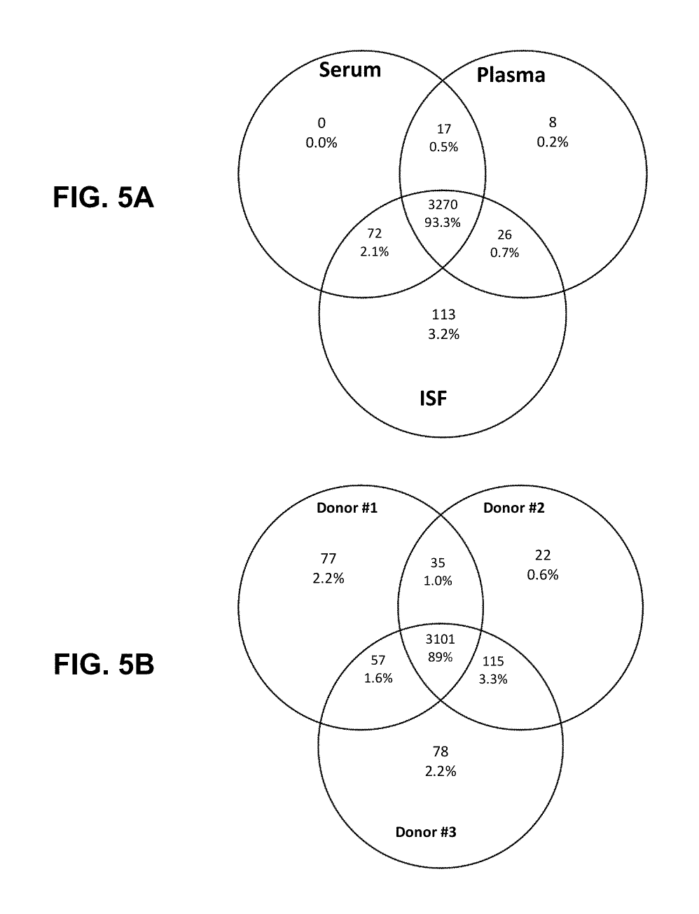

Recently, a detailed study has characterized the proteomic content of dermal ISF using the microneedle array. See Bao Quoc Tran et al., J. Proteome Res. 17, 479 (2018), which is incorporated by reference. In particular, qualitative and quantitative evaluation of the dermal ISF proteome in comparison with patient-matched plasma and serum was used to assess the applicability of microneedle derived ISF as a minimally invasive sampling technique for clinical diagnosis and monitoring. In this study, a microneedle array was used to extract ISF from three healthy human donors, along with matching serum and plasma. The analysis resulted in the identification of 3527 proteins belonging to 1244 protein groups that shared the same set or subset of identified peptides. The Venn diagram in FIG. 5A shows 3270 out of the 3506 proteins identified in donor #1, equivalent to 93%, were ultimately found in common between all biological fluids. Additionally, there was very high overall proteome overlap between all three donors, as shown in FIG. 5B. Similar results were observed for donors #2 and #3 with 95% and 98% proteins in common, respectively. This analysis demonstrated that ISF across all three donors is highly homogeneous and nearly indistinguishable in terms of protein diversity compared with serum and plasma. Most differences were seen at the quantitative level, as the quantity of the top proteins found in ISF differed from those in serum and plasma. Statistical analysis also suggested that ISF is significantly enriched with exosomes compared with serum and plasma. This work suggests that ISF extraction using microneedle arrays is a minimally invasive alternative to serum and plasma, and can be useful for many clinical applications including physiological monitoring and diagnostics. The microneedle array thus provides a sampling foundation for the development of new real-time wearable sensing technologies.

The present invention has been described as a device and method for the extraction of interstitial fluid using microneedle arrays. It will be understood that the above description is merely illustrative of the applications of the principles of the present invention, the scope of which is to be determined by the claims viewed in light of the specification. Other variants and modifications of the invention will be apparent to those of skill in the art.

We claim:

1. A microneedle device for extracting interstitial fluid from an animal, comprising:
a hollow microneedle having a distal end and a beveled tip for penetration of a skin; and an outer holder having an open end with a rim separated from the inner hollow microneedle by an annular open space and wherein the beveled tip of the hollow microneedle protrudes beyond the rim of the outer holder and wherein the rim can press against the skin thereby enabling extraction of interstitial fluid from beneath the skin through the penetrating beveled tip of the hollow microneedle.

2. The microneedle device of claim 1, wherein the outer holder comprises a hollow cylinder with a circular rim.

3. The microneedle device of claim 1, wherein the outer holder has a square or triangular cross section.

4. The microneedle device of claim 1, wherein the hollow microneedle comprises a metal, ceramic, glass, silicon, or polymer.

5. The microneedle device of claim 1, wherein the hollow microneedle comprises a pen needle.

6. The microneedle device of claim 1, wherein the beveled tip of the hollow microneedle protrudes beyond the rim of the outer holder by between 0.5 mm and 2 mm.

7. The microneedle device of claim 1, wherein the outer diameter of the hollow microneedle is less than 400 μm.

8. The microneedle device of claim 2, wherein the inner diameter of the circular rim is between 1 and 5 mm.
